(19) United States
(12) Patent Application Publication
(10) Pub. No.: US 2026/0283788 A1
(43) Pub. Date: Sep. 24, 2026

(54) MANDREL FOR PROSTHETIC HEART VALVE IMPLANT APPARATUS

(71) Applicant: Medtronic, Inc., Minneapolis, MN (US)

(72) Inventors: Matthew C. BALDWIN, Santa Rosa, CA (US); Rodney J. BELL, Newtownabbey Ann (GB); David R. NOLAN, Galway (IE); Yvette C. RAMIREZ, San Francisco, CA (US); Veronica L. WOEN, Westminster, CA (US); Fatemeh FATEMI FAR, Richmond, CA (US); Luanglith BOUNGNASENG, Santa Rosa, CA (US)

(21) Appl. No.: 19/479,091

(22) PCT Filed: Apr. 26, 2024
(86) PCT No.: PCT/US2024/026373

§ 371 Date: Oct. 27, 2025

Related U.S. Application Data
(60) Provisional application No. 63/462,614, filed on Apr. 28, 2023.

(51) Int. Cl. A61F 2/24

(52) U.S. Cl. CPC A61F 2/2415; A61F 2/2418; A61F 2240/005; A61F 2250/001

(57) ABSTRACT

A mandrel imparts a shape to a frame of a heart valve prosthesis. The mandrel includes a body portion extending along a mandrel longitudinal axis between a first end and a second end with a central region extending between the first end and the second end. An outer radial surface is spaced a first radial distance from the mandrel longitudinal axis at a first circumferential location and a second radial distance at a second circumferential location. The first radial distance is different than the second radial distance. The mandrel is received within an inner lumen of the frame. The mandrel includes a cap circumferentially surrounding the second end of the body portion such that the frame is positioned radially between the body portion and the cap. Methods for imparting a shape to an annular frame are provided.

### CROSS-REFERENCE TO RELATED APPLICATIONS

This application claims the benefit of U.S. Provisional Application No. 63/462,614, filed Apr. 28, 2023, the entire contents of which are incorporated herein by reference.

### FIELD

The present disclosure relates generally to a transcatheter heart valve implant apparatus and, more particularly, to a mandrel for imparting a shape to a transcatheter heart valve implant apparatus.

### BACKGROUND

It is known to provide a heart valve implant apparatus for implanting a heart valve prosthesis within a target site of the vasculature of a patient. The heart valve prosthesis can be moved from a radially-collapsed position to a radially-expanded position. The shape of the heart valve prosthesis can impact the performance of the valve and the durability of the valve leaflets. Imparting a desired shape to the heart valve prosthesis may improve performance of the valve and increase durability of the valve leaflets, leading to improved lifetime management.

### SUMMARY

The following presents a simplified summary of the disclosure to provide a basic understanding of some aspects described in the detailed description.

In aspects, a mandrel for imparting a shape to a frame of a heart valve prosthesis is provided. The mandrel comprises a body portion extending along a mandrel longitudinal axis between a first end and a second end with a central region extending between the first end and the second end. An outer radial surface of the central region is spaced a first radial distance from the mandrel longitudinal axis at a first circumferential location and spaced a second radial distance from the mandrel longitudinal axis at a second circumferential location that is circumferentially spaced apart from the first circumferential location. The first radial distance is different than the second radial distance. The mandrel is configured to be received within an inner lumen of the frame. A cap is positioned circumferentially surrounding the second end of the body portion such that the frame is configured to be positioned radially between the body portion and the cap.

In aspects, the central region is spaced a first distance from the first end and a second distance from the second end.

In aspects, the first radial distance is greater than the second radial distance.

In aspects, the body portion comprises, at a first axial location along the mandrel longitudinal axis, a first mandrel extension portion at the first circumferential location, the first mandrel extension portion comprising the first radial distance. A first mandrel intermediate portion is at the second circumferential location. The first mandrel intermediate portion comprises the second radial distance. The first circumferential location and the second circumferential location are spaced circumferentially apart within a range from about 45 degrees to about 75 degrees about the mandrel longitudinal axis.

In aspects, the body portion comprises, at the first axial location along the mandrel longitudinal axis, a second mandrel extension portion at a third circumferential location. The second mandrel extension portion comprises the first radial distance. The second circumferential location and the third circumferential location are spaced circumferentially apart within a range from about 45 degrees to about 75 degrees about the mandrel longitudinal axis. A second mandrel intermediate portion is at a fourth circumferential location. The second mandrel intermediate portion comprises the second radial distance. The third circumferential location and the fourth circumferential location are spaced circumferentially apart within a range from about 45 degrees to about 75 degrees about the mandrel longitudinal axis.

In aspects, the body portion comprises, at the first axial location along the mandrel longitudinal axis, a third mandrel extension portion at a fifth circumferential location. The third mandrel extension portion comprises the first radial distance. The fourth circumferential location and the fifth circumferential location are spaced circumferentially apart within a range from about 45 degrees to about 75 degrees about the mandrel longitudinal axis. A third mandrel intermediate portion is at a sixth circumferential location. The third mandrel intermediate portion comprises the second radial distance. The fifth circumferential location and the sixth circumferential location are spaced circumferentially apart within a range from about 45 degrees to about 75 degrees about the mandrel longitudinal axis. The first circumferential location and the sixth circumferential location are spaced circumferentially apart within a range from about 45 degrees to about 75 degrees about the mandrel longitudinal axis.

In aspects, the cap extends along the mandrel longitudinal axis and comprises one or more projections that project from an end of the cap toward the first end of the body portion.

In aspects, the one or more projections comprise a first projection, a second projection spaced circumferentially apart from the first projection within a range from about 100 degrees to about 140 degrees, and a third projection spaced circumferentially apart from the second projection within a range from about 100 degrees to about 140 degrees.

In aspects, a transcatheter heart valve prosthesis comprises an annular frame extending along a frame longitudinal axis between an inflow end of the transcatheter heart valve prosthesis and an outflow end of the transcatheter heart valve prosthesis. The annular frame comprises a plurality of struts and is configured to be adjustable between a radially-collapsed position and a radially-expanded position. The annular frame comprises a first radius at a first circumferential location and a second radius at a second circumferential location. The first circumferential location and the second circumferential location are located at the same axial location along the frame longitudinal axis between the inflow end and the outflow end, the first radius different than the second radius.

In aspects, the first radius is greater than the second radius.

In aspects, the annular frame comprises, at a first axial location along the frame longitudinal axis, a first frame extension portion at the first circumferential location. The first frame extension portion comprises the first radius. A first frame intermediate portion is at the second circumferential location. The first frame intermediate portion comprises the second radius. The first circumferential location and the second circumferential location are spaced circumferentially apart within a range from about 45 degrees to about 75 degrees about the frame longitudinal axis.

In aspects, the annular frame comprises, at the first axial location along the frame longitudinal axis, a second frame extension portion at a third circumferential location. The second frame extension portion comprises the first radius. The second circumferential location and the third circumferential location are spaced circumferentially apart within a range from about 45 degrees to about 75 degrees about the frame longitudinal axis. A second frame intermediate portion is at a fourth circumferential location. The second frame intermediate portion comprises the second radius. The third circumferential location and the fourth circumferential location are spaced circumferentially apart within a range from about 45 degrees to about 75 degrees about the frame longitudinal axis.

In aspects, the annular frame comprises, at the first axial location along the frame longitudinal axis, a third frame extension portion at a fifth circumferential location. The third frame extension portion comprises the first radius. The fourth circumferential location and the fifth circumferential location are spaced circumferentially apart within a range from about 45 degrees to about 75 degrees about the frame longitudinal axis. A third frame intermediate portion is at a sixth circumferential location. The third frame intermediate portion comprises the second radius. The fifth circumferential location and the sixth circumferential location are spaced circumferentially apart within a range from about 45 degrees to about 75 degrees about the frame longitudinal axis. The first circumferential location and the sixth circumferential location are spaced circumferentially apart within a range from about 45 degrees to about 75 degrees about the frame longitudinal axis.

In aspects, methods for imparting a shape to an annular frame of a heart valve prosthesis comprise positioning a body portion of a mandrel within a lumen of the frame. The body portion extends along a mandrel longitudinal axis between a first end and a second end with a central region extending between the first end and the second end. An outer radial surface of the central region is spaced a first radial distance from the mandrel longitudinal axis at a first location and spaced a second radial distance from the mandrel longitudinal axis at a second location that is spaced circumferentially apart from the first location. The first radial distance is different than the second radial distance. Methods can comprise contacting the frame with the body portion to impart a shape to the frame such that the frame comprises a first radius at a first circumferential location and a second radius at a second circumferential location. The first circumferential location and the second circumferential location are located at the same axial location along a frame longitudinal axis of the frame. The first radius is different than the second radius.

In aspects, methods further comprise positioning a cap of the mandrel radially exterior from the frame such that the cap circumferentially surrounds the frame and the body portion with the frame radially between the cap and the body portion.

In aspects, methods further comprise contacting an outer radial surface of the frame with one or more projections of the cap. The one or more projections projects from an end of the cap toward the first end of the body portion.

In aspects, the one or more projections constrain outer radial movement of portions of the frame in contact with the one or more projections. The one or more projections comprises a first projection, a second projection spaced circumferentially apart from the first projection within a range from about 100 degrees to about 140 degrees, and a third projection spaced circumferentially apart from the second projection within a range from about 100 degrees to about 140 degrees.

In aspects, the first radius is greater than the second radius.

In aspects, contacting the frame with the body portion to impart the shape comprises imparting a plurality of frame extension portions comprising the first radius and a plurality of frame intermediate portions comprising the second radius. The plurality of frame extension portions and the plurality of frame intermediate portions are located at the same axial location along the frame longitudinal axis.

In aspects, a frame extension portion of the plurality of frame extension portions is spaced circumferentially apart from an intermediate portion of the plurality of frame intermediate portions within a range from about 45 degrees to about 75 degrees about the frame longitudinal axis.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates a side view of example aspects of a transcatheter heart valve implant apparatus in accordance with aspects of the disclosure;

FIG. 2 is a top-down image of valve components attached to a frame of the transcatheter heart valve implant apparatus in accordance with aspects of the disclosure;

FIG. 3 schematically illustrates retraction of a capsule of the transcatheter heart valve implant apparatus in accordance with aspects of the disclosure;

FIG. 4 illustrates deployment of a heart valve prosthesis in accordance with aspects of the disclosure;

FIG. 5 schematically illustrates a side view of additional aspects of the delivery of a transcatheter heart valve implant apparatus in accordance with aspects of the disclosure;

FIG. 6 schematically illustrates retraction of a capsule of the transcatheter heart valve implant apparatus in accordance with aspects of the disclosure;

FIG. 7 illustrates a sectional view of the transcatheter heart valve implant apparatus along lines 7-7 of FIG. 1 in accordance with aspects of the disclosure;

FIG. 8 illustrates a mandrel in a disassembled state in accordance with aspects of the disclosure;

FIG. 9 illustrates a mandrel in an assembled state in accordance with aspects of the disclosure;

FIG. 10 illustrates the frame in contact with a body portion of the mandrel in accordance with aspects of the disclosure;

FIG. 11 illustrates a sectional view of the transcatheter heart valve implant apparatus along lines 11-11 of FIG. 10 in accordance with aspects of the disclosure;

FIG. 12 illustrates a mandrel in an assembled state with a cap comprising one or more projections in accordance with aspects of the disclosure;

FIG. 13 illustrates a projection extending from the cap in accordance with aspects of the disclosure;

FIG. 14 illustrates a sectional view of the transcatheter heart valve implant apparatus along lines 14-14 of FIG. 13 in accordance with aspects of the disclosure.

FIG. 15 illustrates a perspective view of a balloon for use with a balloon-expandable heart valve frame in accordance with aspects of the disclosure; and

FIG. 16 illustrates an end view of the balloon along lines 16-16 of FIG. 15 in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein-for example up, down, right, left, front, back, top, bottom, upper, lower, etc.-are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

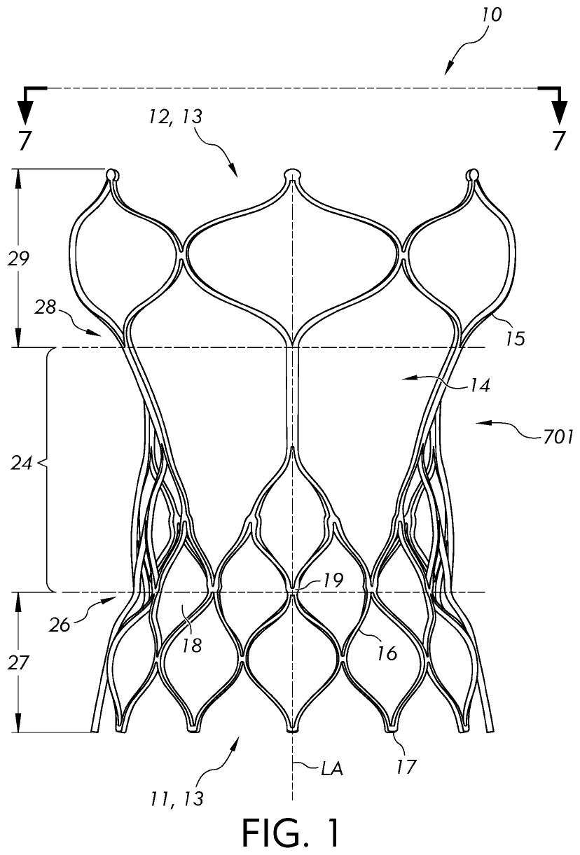

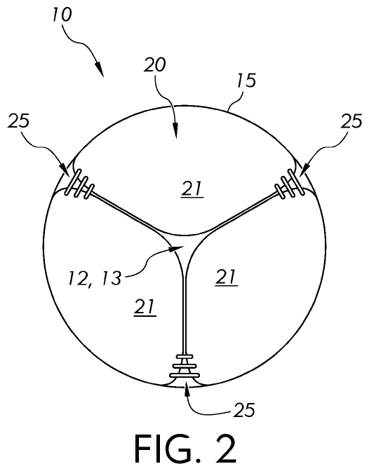

FIGS. 1 and 2 illustrate an example transcatheter heart valve prosthesis 10. The mandrel described herein may be used with the transcatheter heart valve prosthesis 10 and/or other transcatheter heart valve prostheses. The transcatheter heart valve prosthesis 10 is illustrated to facilitate description of the disclosure. The following description of the transcatheter heart valve prosthesis 10 is merely exemplary in nature and is not intended to limit the application and potential uses of the heart valve prosthesis 10.

FIGS. 1 and 2 illustrate a side view and a top (outflow end) view, respectively, of the transcatheter heart valve prosthesis 10. The transcatheter heart valve prosthesis 10 includes a radially-expandable annular frame or stent 15 and a prosthetic valve 20. The annular frame 15 (e.g., “frame”) of the transcatheter heart valve prosthesis 10 supports the prosthetic valve 20 within an interior of the frame 15, for example, with the prosthetic valve 20 (e.g., illustrated in FIG. 2) attached to the frame 15. In the example transcatheter heart valve prosthesis 10 shown in FIGS. 1 and 2, the frame 15 is self-expandable. However, this is not meant to be limiting, and the frame 15 can be balloon-expandable or mechanically expandable in other embodiments. The frame 15 comprises a plurality of struts 16 such that the frame 15 is adjustable between a radially-collapsed position and a radially-expanded position.

The prosthetic valve 20 includes at least one leaflet 21 disposed within and secured to the frame 15. In the embodiments shown in FIGS. 1 and 2, the prosthetic valve 20 includes exactly three leaflets 21, as shown in FIG. 2. However, this is not meant to be limiting, as the prosthetic valve 20 may include more or fewer leaflets 21. The valve leaflets 21 open and close to regulate flow through the transcatheter heart valve prosthesis 10.

As shown in FIG. 1, the transcatheter heart valve prosthesis 10 includes an inflow end 11 and an outflow end 12. The prosthetic leaflets 21 are attached to the frame 15 at commissures 25 such that when pressure at the inflow end 11 exceeds pressure at the outflow end 12, the prosthetic leaflets 21 open to allow blood flow through the heart valve prosthesis 10 from the inflow end 11 to the outflow end 12. When the pressure at the outflow end 12 exceeds pressure at the inflow end 11, the prosthetic leaflets 21 close to prevent blood flow from the outflow end 12 to the inflow end 11. Accordingly, the at least one leaflet (e.g., the prosthetic leaflets 21) can be attached to the plurality of struts 16, for example, by being directly attached to the plurality of struts 16 at the commissures 25, or by being indirectly attached to the plurality of struts 16, for example, by being attached to a skirt, a commissure bracket, or other structure (e.g., mechanical actuator) that is attached to the plurality of struts 16.

The frame 15 of the transcatheter heart valve prosthesis 10 further comprises a plurality of struts 16 that are arranged to form a plurality of openings or cells 18 arranged circumferentially around a frame longitudinal axis LA of the transcatheter heart valve prosthesis 10 and longitudinally to form a tubular structure defining a central lumen 13 of the transcatheter heart valve prosthesis 10. For example, the frame 15 can extend along the frame longitudinal axis LA between the inflow end 11 and the outflow end 12. The frame longitudinal axis LA can extend through a centroid of the frame 15. For example, if the inflow end 11 and the outflow end 12 comprise circular cross-sectional shapes, then the frame longitudinal axis LA can pass through the midpoint, or center, of the circular shape of the inflow end 11 and the outflow end 12. If the inflow end 11 and the outflow end 12 comprise elliptical cross-sectional shapes, then the frame longitudinal axis LA can pass through the midpoint, or center (e.g., of both major and minor axes that are perpendicular at the center), of the elliptical shape of the inflow end 11 and the outflow end 12.

The frame 15 is configured to secure the prosthetic valve 20 within the central lumen 13 of the frame 15 and to secure the transcatheter heart valve prosthesis 10 in place in the vasculature of the patient. The struts 16 are defined herein as the elongated wire segments of the frame 15. Struts 16 come together to form crowns 17 or nodes 19, as can be seen in FIG. 1. The frame 15 of the heart valve prosthesis 10 includes a plurality of cells 18 defined as the spaces between the plurality of crowns 17, the plurality of nodes 19, and the plurality of struts 16. The frame 15, and, thus, the plurality of struts 16, can be adjustable between a radially-collapsed position and a radially-expanded position. The frame 15 can comprise a central frame region 24 that encompasses a center of the frame 15 along the frame longitudinal axis LA. For example, a first end 26 of the central frame region 24 can be spaced a first separating distance 27 from the inflow end 11 and a second end 28 of the central frame region 24 can be spaced a second separating distance 29 from the outflow end 12.

In the example embodiment shown in FIG. 1, the plurality of cells 18 may be diamond-shaped. In the example embodiment shown, the plurality of cells include a plurality of first cells 18 and access cells 14. In particular, the access cells are larger than the first cells 18 and can provide access to one or more coronary arteries when the transcatheter heart valve prosthesis 10 is implanted in the patient. In the embodiment shown, there are exactly six access cells 14. However, this is not meant to be limiting, as the frame 15 of the transcatheter heart valve prosthesis 10 can include more, fewer, or no access cells 14. The access cells 14 each have an enlarged area relative or compared to the first cells 18, as can be seen in FIG. 1. Further, the access cells 14 may be located in other locations than the locations shown in FIG. 1. Although not shown, in some embodiments the transcatheter heart valve prosthesis 10 may include an outer skirt extending circumferentially around an outer circumference of the stent 15 at or near the inflow end 11 to prevent paravalvular leakage of blood around the outside of the transcatheter heart valve prosthesis 10 once implanted in the patient.

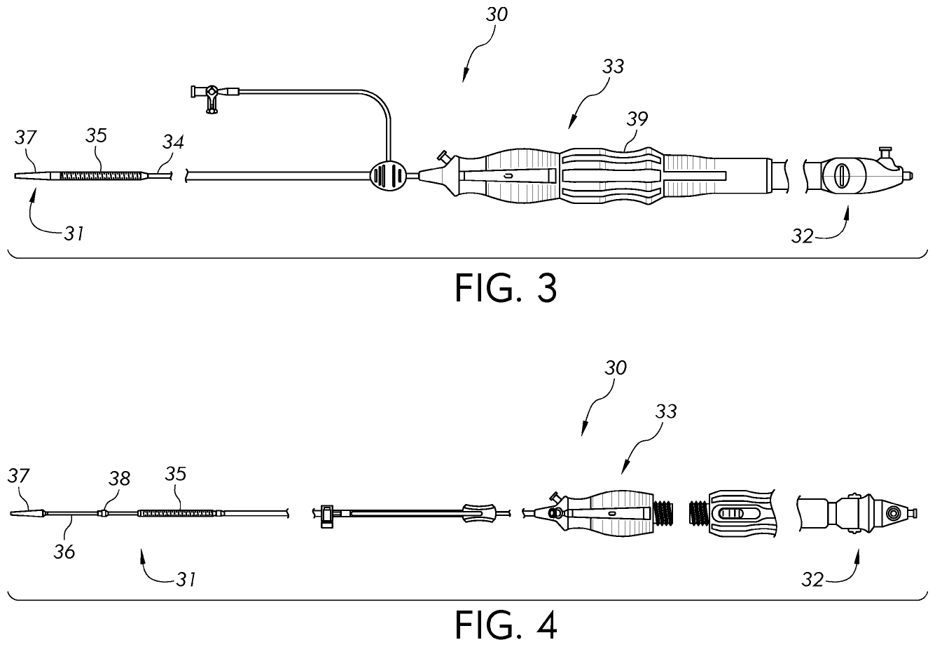

FIGS. 3 and 4 show schematically side views of a delivery assembly 30 for delivering and deploying a transcatheter heart valve prosthesis (e.g., transcatheter heart valve prosthesis 10) according to embodiments hereof. One skilled in the art will realize that FIGS. 3 and 4 illustrate one example of a delivery assembly 30 and that components illustrated in FIGS. 3 and 4 may be removed and/or additional components may be added. The delivery assembly 30 includes a distal end 31, a proximal end 32, and a handle 33. The handle 33 enables a physician to manipulate a distal portion of the delivery assembly 30 and includes actuators for moving parts of the delivery assembly 30 relative to other parts. In the delivery assembly 30, an outer shaft 34 is coupled to an actuator 39 of the handle 33 for moving the outer shaft 34 relative to an inner shaft 36.

A distal portion of the outer shaft 34, referred to as a capsule 35, is configured to surround a transcatheter heart valve prosthesis (e.g., transcatheter heart valve prosthesis 10) during delivery to the treatment site (e.g., a native heart valve) and is retracted from the transcatheter heart valve prosthesis to expose the transcatheter heart valve prosthesis such that it self-expands. The inner shaft 36 is coupled to the handle 33 and movement of the handle 33 translates to movement of the inner shaft 36 and a distal tip or nosecone 37 coupled to a distal end of the inner shaft 36. The inner shaft 36 and distal tip or nosecone 37 may also be translated relative to the outer shaft 34 and the handle 33 via a tip retractor. In the embodiment shown, the inner shaft 36 includes a retainer or spindle 38 for receiving the paddles of the transcatheter heart valve prosthesis 10.

When the actuator 39 is actuated, the actuator 39 moves the outer shaft 34 and the capsule 35 relative to the inner shaft 36, as shown in FIG. 4. As known to those skilled in the art, when the delivery assembly 30 is in position such that the transcatheter heart valve prosthesis 10 is at the desired position at the treatment site in the patient's vasculature, the actuator 39 is actuated to move the capsule 35 relative to the inner shaft 36 and the transcatheter heart valve prosthesis 10 disposed between the inner shaft 36 and the capsule 35, thereby enabling the transcatheter heart valve prosthesis 10 to deploy via self-expansion at the treatment site and release from the spindle 38, as shown in FIG. 4 (without showing the transcatheter heart valve prosthesis 10).

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient.

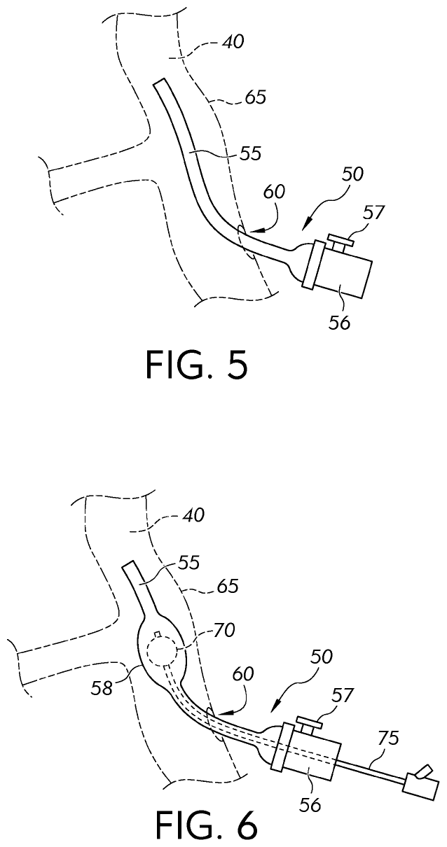

FIGS. 5 and 6 depict one embodiment of an introducer sheath 50 positioned through an incision 60 in the skin 65 of a patient and into a vessel 40 of a patient. The sheath 50 has a tubular shaft 55 and a proximal hub 56 with a hemostatic seal and a luer lock 57. FIG. 5 shows the sheath 50 positioned in the vessel 40 in its normal, unexpanded state, while FIG. 6 shows the sheath 50 positioned in the vessel 40 with a delivery device 75 delivering another device 70 that is being advanced through the sheath 50 such that the tubular shaft 55 expands or deforms at the location where the device 70 is passing through. The shaft 55 expands at expanded region 58 when the device 70 passes through and then retracts or recovers to its original diameter after the device 70 moves past or is removed from the shaft 55. Thus, the tubular shaft 55 is configured to be expandable and retractable.

In certain embodiments, the expandability of the shaft 55 (and any shaft described according to any embodiment set forth herein) is achieved via the elasticity of the shaft 55, which can result in the shaft 55 being either self-expandable or self-expanding or mechanically expandable or mechanically expanding. For purposes of this application, self-expandable means that the shaft 55 is configured to expand to a predetermined or nominal diameter automatically (without any type of actuation, mechanical or otherwise). Further, for purposes of this application, mechanically expandable means that the shaft 55 is configured to expand when a positionable medical device is positioned through the shaft 55. That is, the device itself that is being passed through the shaft 55 causes the expansion of the shaft 55, as depicted in FIG. 6. Alternatively, the expandable characteristics of the shaft 55 can be caused by something other than elasticity.

After passage of the device, the shaft 55 is configured to be contractable, retractable, or recoverable to its original, unexpanded state as depicted in FIG. 5. The retractability can be, in certain embodiments, achieved by the elasticity of the shaft 55, which can result in the shaft 55 being either self-retractable or self-retracting, self-recoverable, or self-contractable, or mechanically retractable or mechanically retracting, mechanically recoverable, or mechanically contractable. For purposes of this application, self-retractable means that the shaft 55 is configured to retract to a predetermined or nominal diameter automatically (without any type of actuation, mechanical or otherwise). Further, for purposes of this application, mechanically retractable means that the shaft 55 is configured to retract when a device or component is used to cause the shaft 55 to retract or recover. Alternatively, the retractable characteristics of the shaft 55 can be caused by something other than elasticity.

For purposes of this application, any device that can be positioned through an introducer sheath according to any embodiment disclosed or contemplated herein can be referred to as a positionable medical device or insertable medical device. Such devices include guidewires, dilators, delivery devices (for delivery and/or placement of structural elements such as heart valves, heart valve repair devices, occluders, grafts, electrical stimulators, leads, etc.), guide catheters, guiding sheaths, diagnostic catheters, stent delivery systems, balloon catheters, and other known vascular devices. Other devices can include non-vascular devices such as scopes and other common surgical instruments. Further, the introducer sheath is configured to receive tissues or organs. Thus, as one non-limiting example, the introducer sheath 50 is described as being an expandable introducer sheath 50 for introduction of a delivery assembly 30 including a transcatheter heart valve prosthesis 10.

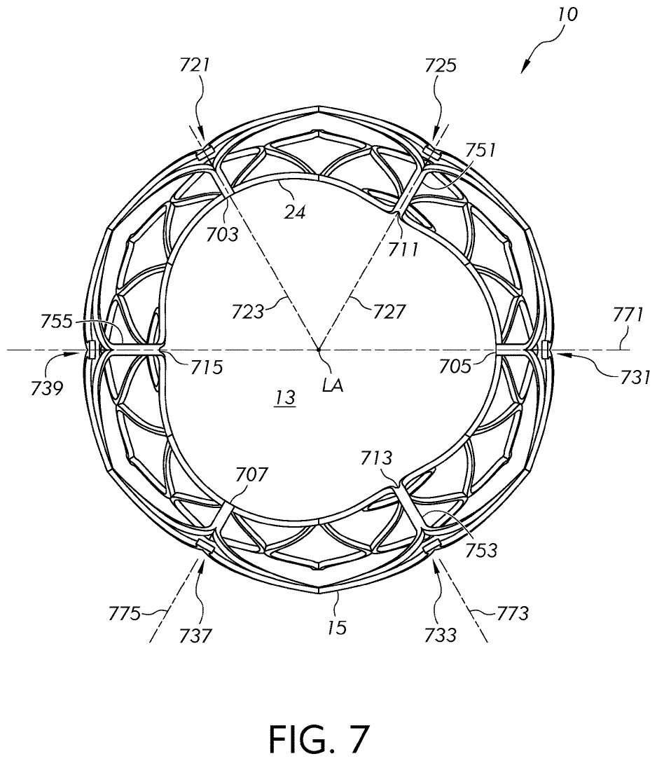

FIG. 7 illustrates a top-down view of the heart valve prosthesis 10 into the outflow end 12 as viewed from the perspective indicated by lines 7-7 of FIG. 1. In aspects, the central frame region 24 can comprise one or more extension portions and one or more intermediate portions located at a first axial location 701 of the central frame region 24. The first axial location 701 is illustrated in FIG. 1 and comprises a location along the frame longitudinal axis LA and within the central frame region 24. In aspects, the central frame region 24 can encompass a midpoint of the frame 15 that is about halfway between the between the inflow end 11 and the outflow end 12. In aspects, the central frame region 24 can encompass the length along the frame longitudinal axis LA at which the leaflets 21 occupy when in the fully opened position and when in the closed position. In aspects, the first axial location 701 can be located at a position occupied by the leaflets 21 when the leaflets 21 are in the fully opened position.

The one or more extension portions of the frame 15 can comprise, at the first axial location 701, a first frame extension portion 703, a second frame extension portion 705, and a third frame extension portion 707. The one or more intermediate portions of the frame 15 can comprise, at the first axial location 701, a first frame intermediate portion 711, a second frame intermediate portion 713, and a third frame intermediate portion 715. The frame extension portions 703, 705, 707 and the frame intermediate portions 711, 713, 715 can be spaced circumferentially apart about the frame longitudinal axis LA, with the frame extension portions 703, 705, 707 in alternating positions with the frame intermediate portions 711, 713, 715. For example, the first frame extension portion 703 is positioned circumferentially between the first frame intermediate portion 711 and the third frame intermediate portion 715. The first frame intermediate portion 711 is positioned circumferentially between the first frame extension portion 703 and the second frame extension portion 705. The second frame extension portion 705 is positioned circumferentially between the first frame intermediate portion 711 and the second frame intermediate portion 713. The second frame intermediate portion 713 is positioned circumferentially between the second frame extension portion 705 and the third frame extension portion 707. The third frame extension portion 707 is positioned circumferentially between the second frame intermediate portion 713 and the third frame intermediate portion 715. The third frame intermediate portion 715 is positioned circumferentially between the third frame extension portion 707 and the first frame extension portion 703.

In aspects, the frame extension portions 703, 705, 707 can comprise a radius (e.g., distance from the frame longitudinal axis LA) to the frame 15 that is different than a radius of the frame intermediate portions 711, 713, 715. For example, the first frame extension portion 703 is positioned at a first circumferential location 721 and comprises a first radius 723. In aspects, the first frame extension portion 703 can comprise a rounded shape with the first radius 723 forming a maximum radius of the first frame extension portion 703, and with radii on opposing circumferential sides of the first circumferential location 721 being less than the first radius 723. The first frame intermediate portion 711 can be positioned at a second circumferential location 725 and may comprise a second radius 727. In aspects, the first circumferential location 721 and the second circumferential location 725 are located at the same axial location (e.g., the first axial location 701) along the frame longitudinal axis LA between the inflow end 11 and the outflow end 12. In aspects, the first frame intermediate portion 711 may be flatter and less rounded than the first frame extension portion 703, with the second radius 727 forming a minimum radius of the first frame intermediate portion 711, and with radii on opposing circumferential sides of the second circumferential location 725 being greater than the second radius 727. In this way, in aspects, the frame extension portions 703, 705, 707 can comprise a different shape than the frame intermediate portions 711, 713, 715. In aspects, the first radius 723 may be different than the second radius 727, for example, with the first radius 723 greater than the second radius 727. In aspects, the difference between the first radius 723 and the second radius 727 may be within a range from about 1 millimeter (“mm”) to about 4 mm, or from about 1.5 mm to about 2.5 mm. In other aspects, the difference between the first radius 723 and the second radius 727 may be about 1.6 mm, or about 2.2 mm. The first circumferential location 721 and the second circumferential location 725 may be spaced circumferentially apart about the frame longitudinal axis LA, for example, within a range from about 45 degrees to about 75 degrees about the frame longitudinal axis LA.

The second frame extension portion 705 is positioned at a third circumferential location 731 and comprises the first radius 723. In aspects, the second frame extension portion 705 can comprise substantially the same shape as the first frame extension portion 703. The second frame intermediate portion 713 can be positioned at a fourth circumferential location 733 and may comprise the second radius 727. In aspects, the second frame intermediate portion 713 can comprise substantially the same shape as the first frame intermediate portion 711. The third circumferential location 731 and the fourth circumferential location 733 are located at the same axial location (e.g., the first axial location 701) along the frame longitudinal axis LA between the inflow end 11 and the outflow end 12. The third circumferential location 731 and the fourth circumferential location 733 may be spaced circumferentially apart about the frame longitudinal axis LA, for example, within a range from about 45 degrees to about 75 degrees about the frame longitudinal axis LA.

The third frame extension portion 707 is positioned at a fifth circumferential location 737 and comprises the first radius 723. In aspects, the third frame extension portion 707 can comprise substantially the same shape as the first frame extension portion 703. The third frame intermediate portion 715 can be positioned at a sixth circumferential location 739 and may comprise the second radius 727. In aspects, the third frame intermediate portion 715 can comprise substantially the same shape as the first frame intermediate portion 711. The fifth circumferential location 737 and the sixth circumferential location 739 are located at the same axial location (e.g., the first axial location 701) along the frame longitudinal axis LA between the inflow end 11 and the outflow end 12. The fifth circumferential location 737 and the sixth circumferential location 739 may be spaced circumferentially apart about the frame longitudinal axis LA, for example, within a range from about 45 degrees to about 75 degrees about the frame longitudinal axis LA. In aspects, the first circumferential location 721 and the sixth circumferential location 739 may be spaced circumferentially apart about the frame longitudinal axis LA, for example, within a range from about 45 degrees to about 75 degrees about the frame longitudinal axis LA.

In aspects, the locations of the frame intermediate portions 711, 713, 715 can substantially match locations of commissures (e.g., substantially identical in size, shape, position, and function to the commissures 25 illustrated in FIG. 2) of the frame 15. For example, the frame 15 can comprise a first commissure 751 that is located substantially at the second circumferential location 725, for example, aligned with the first frame intermediate portion 711. The frame 15 can comprise a second commissure 753 that is located substantially at the fourth circumferential location 733, for example, aligned with the second frame intermediate portion 713. The frame 15 can comprise a third commissure 755 that is located substantially at the sixth circumferential location 739, for example, aligned with the third frame intermediate portion 715. In aspects, a first axis 771 may be perpendicular to the frame longitudinal axis LA, with the first axis 771 intersecting the frame longitudinal axis LA, the second frame extension portion 705 at the third circumferential location 731, and the third frame intermediate portion 715 at the sixth circumferential location 739. In aspects, a second axis 773 may be perpendicular to the frame longitudinal axis LA, with the second axis 773 intersecting the frame longitudinal axis LA, the first frame extension portion 703 at the first circumferential location 721, and the second frame intermediate portion 713 at the fourth circumferential location 733. In aspects, a third axis 775 may be perpendicular to the frame longitudinal axis LA, with the third axis 775 intersecting the frame longitudinal axis LA, the third frame extension portion 707 at the fifth circumferential location 737, and the first frame intermediate portion 711 at the second circumferential location 725.

In some aspects, the central frame region 24 can comprise a substantially constant shape and size along the length of the central frame region 24 between the first end 26 and the second end 28. That is, by comprising a substantially constant shape and size, the frame extension portions 703, 705, 707 may be spaced substantially the same radial distance (e.g., and at the same circumferential location) from the frame longitudinal axis LA along the length of the central frame region 24, and the frame intermediate portions 711, 713, 715 may be spaced substantially the same radial distance (e.g., and at the same circumferential location) from the frame longitudinal axis LA along the length of the central frame region 24. In this way, other axial locations along the frame longitudinal axis LA of the central frame region 24 (e.g., different axial locations from the first axial location 701) can comprise frame extension portions that are substantially identical in shape, size, and position to the frame extension portions 703, 705, 707 at the first axial location 701 and can comprise frame intermediate portions that are substantially identical in shape, size, and position to the frame intermediate portions 711, 713, 715 at the first axial location 701. However, in other aspects, the central frame region 24 may not comprise a substantially constant shape and/or size along the length of the central frame region 24 between the first end 26 and the second end 28.

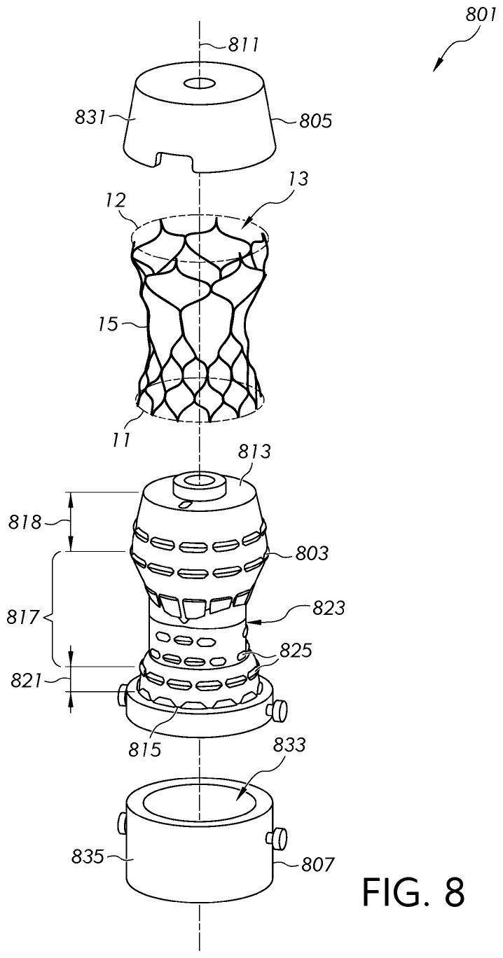

FIG. 8 illustrates a mandrel 801 for imparting a shape (e.g., the shape illustrated and described relative to FIG. 7) to the frame 15 of the heart valve prosthesis 10. The mandrel 801 can comprise a body portion 803 and one or more caps, for example, a first cap 805 and a second cap 807. The frame 15 is illustrated schematically to show that the frame 15 may initially comprise a cylindrical shape or other shape different than the shape illustrated in FIG. 7 prior to contacting the mandrel 801. For example, as noted, by employing the mandrel 801, mechanical memory may be imparted to the frame 15 by thermal treatment to achieve a spring temper or to set a shape memory. Thus, the devices and methods of the present disclosure provide, alone or in combination, a mandrel 801 for imparting a shape to the frame 15 and a frame 15 having a shape imparted thereon by the mandrel 801.

The body portion 803 can extend along a mandrel longitudinal axis 811 between a first end 813 and a second end 815. The body portion 803 can comprise a central region 817 spaced a first distance 818 from the first end 813 and spaced a second distance 821 from the second end 815. In aspects, the central region 817 of the body portion 803 can substantially match a size and location of the central frame region 24 when the frame 15 is in contact with the body portion 803. The body portion 803 can comprise an outer radial surface 823 that circumferentially surrounds, and extends along, the mandrel longitudinal axis 811. The outer radial surface 823 can form several shapes based on a desired shape of the frame 15, and in FIG. 8, the outer radial surface 823 can form an hourglass shape with a non-constant cross-sectional size along the mandrel longitudinal axis 811. The body portion 803 can comprise one or more projections 825 that project radially outwardly from the outer radial surface 823, with the one or more projections 825 defining outcroppings, protuberances, extensions, etc. that can be received within the cells 18 of the frame 15. In this way, the projections 825 can limit the likelihood of the frame 15 shifting or moving unintendedly relative to the body portion 803.

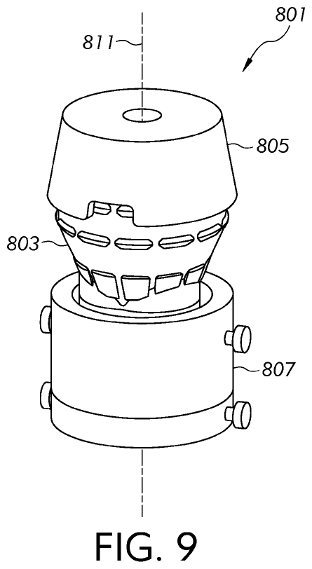

The mandrel 801 comprises the first cap 805 that can be positioned to circumferentially surround the first end 813 of the body portion 803, and the second cap 807 that can be positioned to circumferentially surround the second end 815 of the body portion 803. In aspects, the first cap 805 can comprise a recess (not shown in FIG. 8) that is sized to receive the first end 813. The first cap 805 can comprise a surrounding wall 831 that can circumferentially surround the first end 813. In this way, the outflow end 12 of the frame 15 can be received radially between the surrounding wall 831 of the first cap 805 and the first end 813 of the body portion 803. The frame 15 can contact the body portion 803 such that the first end 813 of the body portion 803 can impart a shape to the outflow end 12 of the frame 15. The second cap 807 can comprise a recess 833 that is sized to receive the second end 815. The second cap 807 can comprise a surrounding wall 835 that can circumferentially surround the second end 815. In this way, the inflow end 11 of the frame 15 can be received radially between the surrounding wall 835 of the second cap 807 and the second end 815 of the body portion 803. The frame 15 can contact the body portion 803 such that the second end 815 of the body portion 803 can impart a shape to the inflow end 11 of the frame 15. In this way, methods for imparting a shape to the frame 15 of the heart valve prosthesis 10 can comprise positioning the body portion 803 of the mandrel 801 within the lumen 13 of the frame 15. FIG. 9 illustrates the mandrel 801, with the body portion 803 received within the first cap 805 and the second cap 807. Accordingly, methods can comprise positioning the second cap 807 of the mandrel 801 radially exterior from the frame 15 such that the second cap 807 circumferentially surrounds the frame 15 and the body portion 803 with the frame 15 radially between the second cap 807 and the body portion 803.

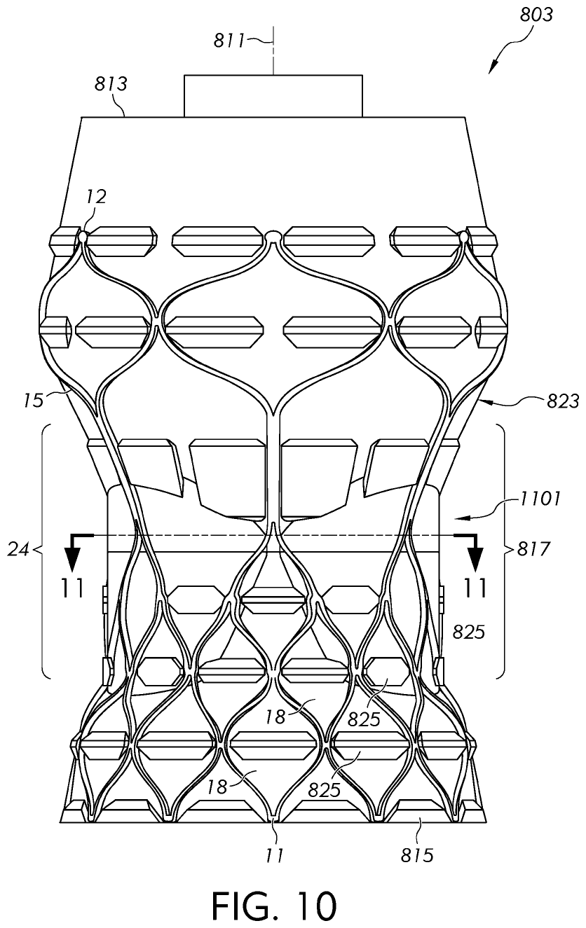

FIG. 10 illustrates the body portion 803 in contact with the frame 15 such that the frame 15 has taken a shape that substantially matches a shape of the body portion 803. For purposes of illustration and to not obstruct portions of the body portion 803 and the frame 15 from view, the mandrel 801 is illustrated without the first cap 805 and the second cap 807. However, in operation, the first cap 805 and the second cap 807 may be positioned in relation to the body portion 803 and the frame 15 in substantially the same manner as illustrated in FIG. 9. In this way, the first cap 805 can be positioned to circumferentially surround the first end 813 of the body portion 803 such that the frame 15 can be positioned radially between the body portion 803 and the first cap 805. Likewise, the second cap 807 can be positioned to circumferentially surround the second end 815 of the body portion 803 such that the frame 15 can be positioned radially between the body portion 803 and the second cap 807. As illustrated in FIG. 10, the frame 15 can be positioned in contact with the body portion 803 such that the projections 825 can extend through the cells 18. The central frame region 24 is in contact with the central region 817 of the body portion 803. For example, the body portion 803 of the mandrel 801 can be received within the lumen 13 of the frame 15. Accordingly, methods can comprise contacting the frame 15 with the body portion 803 to impart a shape to the frame 15 such that the frame 15 comprises the first radius 723 at the first circumferential location 721 and the second radius 727 at the second circumferential location 725. In aspects, contacting the frame 15 with the body portion 803 to impart the shape can comprise imparting the plurality of frame extension portions 703, 705, 707 (e.g., comprising the first radius 723) and the plurality of frame intermediate portions 711, 713, 715 (e.g., comprising the second radius 727).

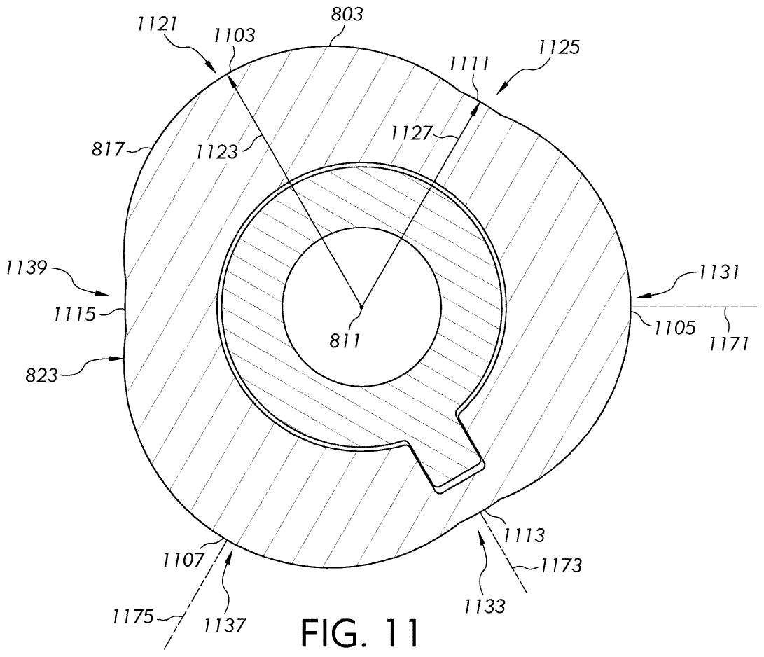

FIG. 11 illustrates a sectional view of the body portion 803 along lines 11-11 of FIG. 10. In aspects, the central region 817 can comprise one or more extension portions and one or more intermediate portions located at a first axial location 1101 (e.g., illustrated in FIG. 10) of the central region 817. The first axial location 1101 comprises a location along the mandrel longitudinal axis 811 and within the central region 817.

The one or more extension portions of the body portion 803 can comprise, at the first axial location 1101, a first mandrel extension portion 1103, a second mandrel extension portion 1105, and a third mandrel extension portion 1107. The one or more mandrel intermediate portions of the body portion 803 can comprise, at the first axial location 1101, a first mandrel intermediate portion 1111, a second mandrel intermediate portion 1113, and a third mandrel intermediate portion 1115. The mandrel extension portions 1103, 1105, 1107 and the mandrel intermediate portions 1111, 1113, 115 can be spaced circumferentially apart about the mandrel longitudinal axis 811, with the mandrel extension portions 1103, 1105, 1107 in alternating positions with the mandrel intermediate portions 1111, 1113, 1115. For example, the first mandrel extension portion 1103 is positioned circumferentially between the first mandrel intermediate portion 1111 and the third mandrel intermediate portion 1115. The first mandrel intermediate portion 1111 is positioned circumferentially between the first mandrel extension portion 1103 and the second mandrel extension portion 1105. The second mandrel extension portion 1105 is positioned circumferentially between the first mandrel intermediate portion 1111 and the second mandrel intermediate portion 1113. The second mandrel intermediate portion 1113 is positioned circumferentially between the second mandrel extension portion 1105 and the third mandrel extension portion 1107. The third mandrel extension portion 1107 is positioned circumferentially between the second mandrel intermediate portion 1113 and the third mandrel intermediate portion 1115. The third mandrel intermediate portion 1115 is positioned circumferentially between the third mandrel extension portion 1107 and the first mandrel extension portion 1103.

In aspects, the mandrel extension portions 1103, 1105, 1107 can comprise a radius (e.g., radial distance from the mandrel longitudinal axis 811) that is different than a radius of the mandrel intermediate portions 1111, 1113, 1115. For example, the first mandrel extension portion 1103 is positioned at a first circumferential location 1121, with the outer radial surface 823 spaced a first radial distance 1123 from the mandrel longitudinal axis 811 at the first circumferential location 1121. In aspects, the first mandrel extension portion 1103 can comprise a rounded shape with the first radial distance 1123 forming a maximum radius of the first mandrel extension portion 1103, and with radii on opposing circumferential sides of the first circumferential location 1121 being less than the first radial distance 1123. The first mandrel intermediate portion 1111 can be positioned at a second circumferential location 1125, with the outer radial surface 823 spaced a second radial distance 1127 from the mandrel longitudinal axis 811 at the second circumferential location 1125. In aspects, the first circumferential location 1121 and the second circumferential location 1125 are located at the same axial location (e.g., the first axial location 1101) along the mandrel longitudinal axis 811. In aspects, the first mandrel intermediate portion 1111 may be flatter and less rounded than the first mandrel extension portion 1103, with the second radial distance 1127 forming a minimum radius of the first mandrel intermediate portion 1111, and with radii on opposing circumferential sides of the second circumferential location 1125 being greater than the second radial distance 1127. In aspects, the first radial distance 1123 may be different than the second radial distance 1127, for example, with the first radial distance 1123 greater than the second radial distance 1127. In aspects, the difference between the first radial distance 1123 and the second radial distance 1127 may be within a range from about 1 mm to about 4 mm, or from about 1.5 mm to about 2.5 mm. In other aspects, the difference between the first radial distance 1123 and the second radial distance 1127 may be about 1.6 mm, or about 2.2 mm. The first circumferential location 1121 and the second circumferential location 1125 may be spaced circumferentially apart about the mandrel longitudinal axis 811, for example, within a range from about 45 degrees to about 75 degrees about the mandrel longitudinal axis 811.

The second mandrel extension portion 1105 is positioned at a third circumferential location 1131 and comprises the first radial distance 1123. In aspects, the second mandrel extension portion 1105 can comprise substantially the same shape as the first mandrel extension portion 1103. The second mandrel intermediate portion 1113 can be positioned at a fourth circumferential location 1133 and may comprise the second radial distance 1127. In aspects, the second mandrel intermediate portion 1113 can comprise substantially the same shape as the first mandrel intermediate portion 1111. The third circumferential location 1131 and the fourth circumferential location 1133 are located at the same axial location (e.g., the first axial location 1101) along the mandrel longitudinal axis 811. The third circumferential location 1131 and the fourth circumferential location 1133 may be spaced circumferentially apart about the mandrel longitudinal axis 811, for example, within a range from about 45 degrees to about 75 degrees about the mandrel longitudinal axis 811.

The third mandrel extension portion 1107 is positioned at a fifth circumferential location 1137 and comprises the first radial distance 1123. In aspects, the third mandrel extension portion 1107 can comprise substantially the same shape as the first mandrel extension portion 1103. The third mandrel intermediate portion 1115 can be positioned at a sixth circumferential location 1139 and may comprise the second radial distance 1127. In aspects, the third mandrel intermediate portion 1115 can comprise substantially the same shape as the first mandrel intermediate portion 1111. The fifth circumferential location 1137 and the sixth circumferential location 1139 are located at the same axial location (e.g., the first axial location 1101) along the mandrel longitudinal axis 811. The fifth circumferential location 1137 and the sixth circumferential location 1139 may be spaced circumferentially apart about the mandrel longitudinal axis 811, for example, within a range from about 45 degrees to about 75 degrees about the mandrel longitudinal axis 811. In aspects, the first circumferential location 1121 and the sixth circumferential location 1139 may be spaced circumferentially apart about the mandrel longitudinal axis 811, for example, within a range from about 45 degrees to about 75 degrees about the mandrel longitudinal axis 811.

In aspects, the locations of the mandrel intermediate portions 1111, 1113, 1115 can impart matching shapes in the frame 15 to form the frame intermediate portions 711, 713, 715 at corresponding locations. Likewise, the locations of the mandrel extension portions 1103, 1105, 1107 can impart matching shapes in the frame 15 to form the frame extension portions 703, 705, 707. For example, the frame 15 can be radially compressed into contact with the outer radial surface 823 of the body portion 803, thus causing the frame 15 to adopt the shape of the outer radial surface 823 of the body portion 803. In aspects, a first axis 1171 may be perpendicular to the mandrel longitudinal axis 811, with the first axis 1171 intersecting the mandrel longitudinal axis 811, the second mandrel extension portion 1105 at the third circumferential location 1131, and the third mandrel intermediate portion 1115 at the sixth circumferential location 1139. In aspects, a second axis 1173 may be perpendicular to the mandrel longitudinal axis 811, with the second axis 1173 intersecting the mandrel longitudinal axis 811, the first mandrel extension portion 1103 at the first circumferential location 1121, and the second mandrel intermediate portion 1113 at the fourth circumferential location 1133. In aspects, a third axis 1175 may be perpendicular to the mandrel longitudinal axis 811, with the third axis 1175 intersecting the mandrel longitudinal axis 811, the third mandrel extension portion 1107 at the fifth circumferential location 1137, and the first mandrel intermediate portion 1111 at the second circumferential location 1125.

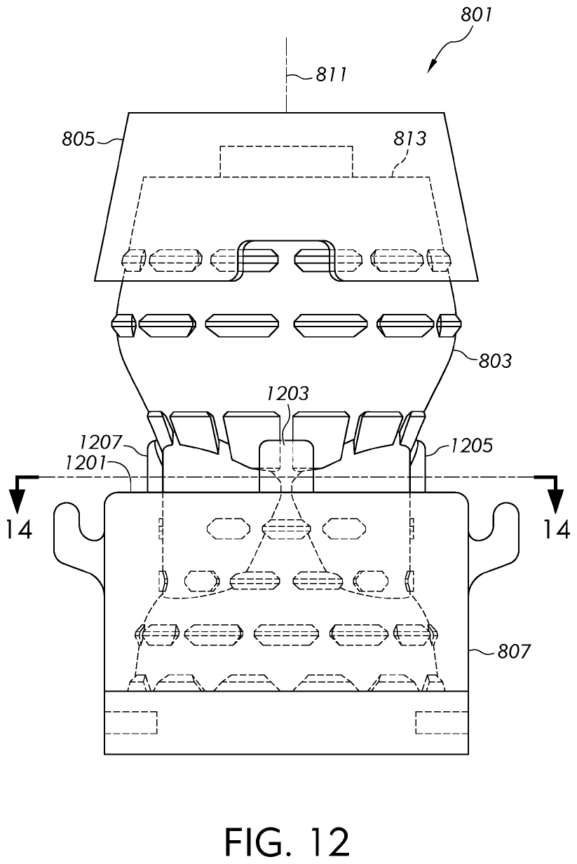

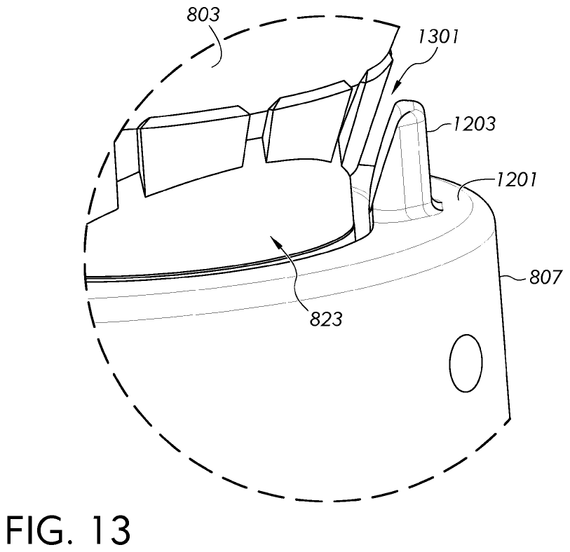

FIG. 12 illustrates the mandrel 801 with the first cap 805 and the second cap 807 receiving the body portion 803. In aspects, the second cap 807 extends along the mandrel longitudinal axis 811 and comprises one or more projections that project from an end 1201 of the second cap 807 toward the first end 813 of the body portion 803. For example, the one or more projections can comprise a first projection 1203, a second projection 1205, and a third projection 1207. FIG. 13 illustrates a close-up enlarged view of the first projection 1203 positioned relative to the outer radial surface 823 of the body portion 803. For example, the first projection 1203 can be spaced apart from the outer radial surface 823 of the central region 817 to define a gap 1301 between the first projection 1203 and the outer radial surface 823. In aspects, gaps that are substantially identical to the gap 1301 can be defined between the second projection 1205 and the outer radial surface 823, and between the third projection 1207 and the outer radial surface 823.

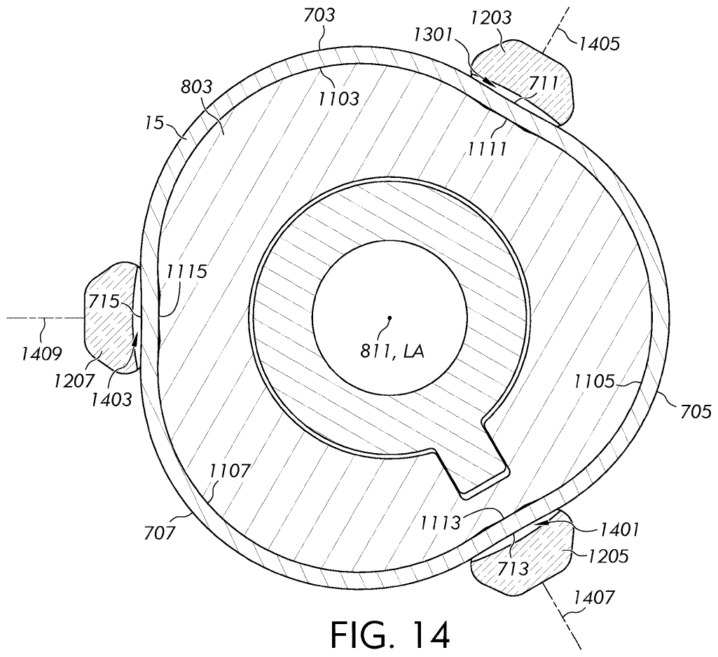

FIG. 14 illustrates a sectional view of the body portion 803, the frame 15, and the projections 1203, 1205, 1207 along lines 14-14 of FIG. 12. For example, a second gap 1401 can be defined between the second projection 1205 and the outer radial surface 823, and a third gap 1403 can be defined between the third projection 1207 and the outer radial surface 823. It will be appreciated that FIG. 14 is merely exemplary and not necessarily drawn to scale, such that the gaps 1301, 1401, 1403, etc. can be larger or smaller in size than as shown. For example, in aspects, each of the gaps 1301, 1401, 1403 can be within a range from 0.6 millimeters to about 0.7 millimeters, though, other gaps sizes are envisioned based on the thickness of the tubing that is used to form the frame.

In aspects, the projections 1203, 1205, 1207 can be positioned at substantially the same circumferential locations as the frame intermediate portions 711, 713, 715 and the mandrel intermediate portions 1111, 1113, 1115. For example, a first radial axis 1405 can intersect the first mandrel intermediate portion 1111, the first frame intermediate portion 711, and the first projection 1203. A second radial axis 1407 can intersect the second mandrel intermediate portion 1113, the second frame intermediate portion 713, and the second projection 1205. A third radial axis 1409 can intersect the third mandrel intermediate portion 1115, the third frame intermediate portion 715, and the third projection 1207. In this way, the first frame intermediate portion 711 can be positioned radially between the first mandrel intermediate portion 1111 and the first projection 1203. The second frame intermediate portion 713 can be positioned radially between the second mandrel intermediate portion 1113 and the second projection 1205. The third frame intermediate portion 715 can be positioned radially between the third mandrel intermediate portion 1115 and the third projection 1207. In aspects, the second projection 1205 is spaced circumferentially apart from the first projection 1203 within a range from about 100 degrees to about 140 degrees. The third projection 1207 can be spaced circumferentially apart from the second projection 1205 within a range from about 100 degrees to about 140 degrees. The third projection 1207 can be spaced circumferentially apart from the first projection 1203 within a range from about 100 degrees to about 140 degrees. In aspects, while the aforementioned projections can be spaced apart within a range from about 100 degrees to about 140 degrees, a maximum radial increase may be spaced about 120 degrees apart.

The projections 1203, 1205, 1207 can limit outward radial expansion of the frame intermediate portions 711, 713, 715 and maintain the frame intermediate portions 711, 713, 715 in proximity to, for example, in contact with, the mandrel intermediate portions 1111, 1113, 1115. In this way, the projections 1203, 1205, 1207 can constrain outer radial movement of the portions of the frame 15 (e.g., frame intermediate portions 711, 713, 715) in contact with the projections 1203, 1205, 1207. Referring to FIGS. 1-14, the shape of the frame 15 described herein can yield several benefits. For example, due to the frame 15 comprising the non-circular shape with the non-constant radius at the central frame region 24, the likelihood of the leaflets 21 inadvertently contacting the frame 15 when the leaflets 21 are in the fully opened position can be reduced. For example, as illustrated in FIG. 7, the location of the commissures 751, 753, 755 can comprise a reduced radius as compared to areas of the frame 15 that are circumferentially between the commissures 751, 753, 755. Accordingly, when the leaflets 21 are in the fully opened position, the leaflets 21 are less likely to contact the frame 15 at the regions with an enlarged radius (e.g., at the frame extension portions 703, 705, 707). Without intending to be bound by theory, it is believed that, as compared to frames having other shapes (e.g., circular cross-section) against which the leaflets 21 may be more likely to contact, the frame 15 of the present disclosure having the regions with an enlarged radius (e.g., at the frame extension portions 703, 705, 707) reduces the likelihood of the leaflets 21 from abrading the frame 15, thereby prolonging the usable, functional life and improving lifetime management of the valve prosthesis 10.

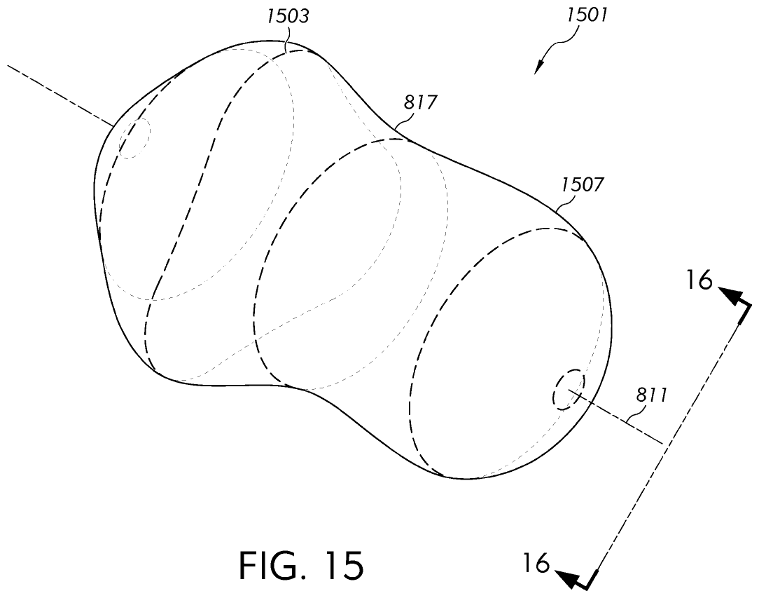

FIG. 15 illustrates a balloon 1501 for radially-expanding the frame 15 when the frame 15 is not a self-expanding frame 15. For example, the frame 15 can comprise a material that is balloon-expandable, and the balloon 1501 can be used to engage the frame 15 and impart the shape to the frame 15. In this way, the frame 15 can have the shape imparted by the balloon 1501, as opposed to the mandrel 801. The balloon 1501 can comprise a material that is capable of being inflated, such as, for example, a nylon material. The balloon 1501 can be in fluid communication with a conduit (e.g., pipe, tube, etc.) that is in fluid communication with a fluid source. In this way, the fluid source can deliver a fluid (e.g., a gas, etc.) through the conduit and to the balloon 1501, such that the balloon 1501 can be inflated and radially-expanded. The balloon 1501 can apply a radial force to the frame 15 and cause the frame 15 to radially-expand with the shape of the balloon 1501 being imparted to the frame 15.

As illustrated in FIG. 15, the balloon 1501 can comprise a plurality of regions, such as, for example, a first region 1503, a second region 1507, and the central region 817 between the first region 1503 and the second region 1507. The first region 1503, the second region 1507, and the central region 817 can be arranged to extend along the longitudinal axis 811. In aspects, the second region 1507 can comprise a rounded or circular shape with a substantially constant radius about the longitudinal axis 811. Likewise, in aspects, the central region 817 can comprise a rounded or circular shape with a substantially constant radius about the longitudinal axis 811. The first region 1503 can comprise a shape that comprises one or more lobes or extension portions (e.g., similar to the shape illustrated in FIG. 11). In this way, the first region 1503 can contact/engage the frame 15 and impart a corresponding shape to the frame 15.

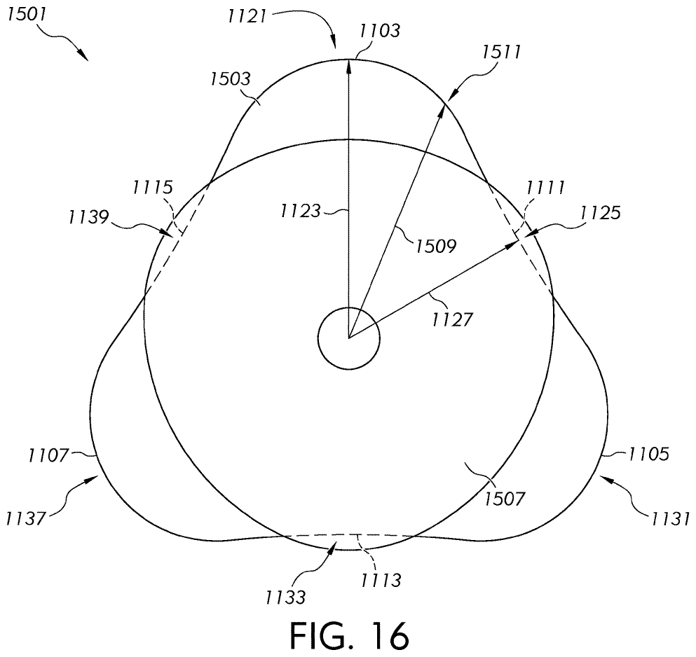

FIG. 16 illustrates an end view of the balloon 1501 along lines 16-16 of FIG. 15. As illustrated, the second region 1507 is in the foreground and comprises the rounded or circular shape. The first region 1503 is in the background (e.g., behind the second region 1507) and comprises the shape with one or more lobes or extension portions. Portions of the first region 1503 are illustrated with dashed lines (e.g., the intermediate portions 1111, 1113, 1115) due to being obstructed from view by the second region 1507.

In aspects, the first region 1503 can comprise a shape that is similar in some respects to the shape of the body portion 803 of the mandrel 801 illustrated in FIG. 11. For example, the first region 1503 can comprise the first extension portion 1103 at the first circumferential location 1121, the first intermediate portion 1111 at the second circumferential location 1125, the second extension portion 1105 at the third circumferential location 1131, the second intermediate portion 1113 at the fourth circumferential location 1133, the third extension portion 1107 at the fifth circumferential location 1137, and the third intermediate portion 1115 at the sixth circumferential location 1139. In aspects, the extension portions 1103, 1105, 1107 can comprise the first radial distance 1123 while the intermediate portions 1111, 1113, 1115 can comprise the second radial distance 1127. In aspects, the first region 1503 can comprise a third radial distance 1509 at a location 1511 that is angularly offset from the first circumferential location 1121. In aspects, the angle between the first circumferential location 1121 and the location 1511 is within a range from about 20 degrees to about 40 degrees, or about 30 degrees. In aspects, the first radial distance 1123 may be up to about 20 millimeters, or up to about 18 millimeters, or up to about 15 millimeters. In aspects, the second radial distance 1127 may be up to about 15 millimeters, or up to about 13 millimeters, or up to about 12.5 millimeters, or up to about 10 millimeters. In aspects, the third radial distance 1509 may be up to about 18 millimeters, or up to about 15 millimeters, or up to about 12 millimeters. It will be appreciated that the dimensions listed herein are merely exemplary, and that other dimensions are possible depending on, for example, the size of the frame 15, the size of the leaflets, etc.

Accordingly, when the balloon 1501 is radially-expanded due to inflation of the balloon 1501, the balloon 1501 can contact the frame 15 (e.g., due to the balloon 1501 being received within a lumen of the frame 15) and impart a shape to the frame 15, wherein the shape can be substantially similar or identical to the shapes of the first region 1503, the second region 1507, and the central region 817. As such, the second region 1507 can impart the shape (e.g., of the second region 1507) to the inflow end 11 of the frame 15 while the first region 1503 can impart the shape (e.g., of the first region 1503) to the outflow end 12 of the frame 15.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A mandrel for imparting a shape to a frame of a heart valve prosthesis, the mandrel comprising:
a body portion extending along a mandrel longitudinal axis between a first end and a second end with a central region extending between the first end and the second end, an outer radial surface of the central region spaced a first radial distance from the mandrel longitudinal axis at a first circumferential location and spaced a second radial distance from the mandrel longitudinal axis at a second circumferential location that is circumferentially spaced apart from the first circumferential location, the first radial distance different than the second radial distance, the mandrel configured to be received within an inner lumen of the frame; and
a cap positioned circumferentially surrounding the second end of the body portion such that the frame is configured to be positioned radially between the body portion and the cap.

2. The mandrel of claim 1, wherein the central region is spaced a first distance from the first end and a second distance from the second end.

3. The mandrel of claim 1, wherein the first radial distance is greater than the second radial distance.

4. The mandrel of claim 3, wherein the body portion comprises, at a first axial location along the mandrel longitudinal axis:
a first mandrel extension portion at the first circumferential location, the first mandrel extension portion comprising the first radial distance; and
a first mandrel intermediate portion at the second circumferential location, the first mandrel intermediate portion comprising the second radial distance, and wherein the first circumferential location and the second circumferential location are spaced circumferentially apart within a range from about 45 degrees to about 75 degrees about the mandrel longitudinal axis.

5. The mandrel of claim 4, wherein the body portion comprises, at the first axial location along the mandrel longitudinal axis:
a second mandrel extension portion at a third circumferential location, the second mandrel extension portion comprising the first radial distance, and wherein the second circumferential location and the third circumferential location are spaced circumferentially apart within a range from about 45 degrees to about 75 degrees about the mandrel longitudinal axis; and
a second mandrel intermediate portion at a fourth circumferential location, the second mandrel intermediate portion comprising the second radial distance, and wherein the third circumferential location and the fourth circumferential location are spaced circumferentially apart within a range from about 45 degrees to about 75 degrees about the mandrel longitudinal axis.

6. The mandrel of claim 5, wherein the body portion comprises, at the first axial location along the mandrel longitudinal axis:
a third mandrel extension portion at a fifth circumferential location, the third mandrel extension portion comprising the first radial distance, and wherein the fourth circumferential location and the fifth circumferential location are spaced circumferentially apart within a range from about 45 degrees to about 75 degrees about the mandrel longitudinal axis; and
a third mandrel intermediate portion at a sixth circumferential location, the third mandrel intermediate portion comprising the second radial distance, and wherein the fifth circumferential location and the sixth circumferential location are spaced circumferentially apart within a range from about 45 degrees to about 75 degrees about the mandrel longitudinal axis, and wherein the first circumferential location and the sixth circumferential location are spaced circumferentially apart within a range from about 45 degrees to about 75 degrees about the mandrel longitudinal axis.

7. The mandrel of claim 1, wherein the cap extends along the mandrel longitudinal axis and comprises one or more projections that project from an end of the cap toward the first end of the body portion.

8. The mandrel of claim 7, wherein the one or more projections comprise:
a first projection;
a second projection spaced circumferentially apart from the first projection within a range from about 100 degrees to about 140 degrees; and
a third projection spaced circumferentially apart from the second projection within a range from about 100 degrees to about 140 degrees.

9. A transcatheter heart valve prosthesis comprising:
an annular frame extending along a frame longitudinal axis between an inflow end of the transcatheter heart valve prosthesis and an outflow end of the transcatheter heart valve prosthesis, the annular frame comprising a plurality of struts and configured to be adjustable between a radially-collapsed position and a radially-expanded position, the annular frame comprising a first radius at a first circumferential location and a second radius at a second circumferential location, the first circumferential location and the second circumferential location located at the same axial location along the frame longitudinal axis between the inflow end and the outflow end, the first radius different than the second radius.

10. The mandrel of claim 9, wherein the first radius is greater than the second radius.

11. The heart valve prosthesis of claim 10, wherein the annular frame comprises, at a first axial location along the frame longitudinal axis:
a first frame extension portion at the first circumferential location, the first frame extension portion comprising the first radius; and
a first frame intermediate portion at the second circumferential location, the first frame intermediate portion comprising the second radius, and wherein the first circumferential location and the second circumferential location are spaced circumferentially apart within a range from about 45 degrees to about 75 degrees about the frame longitudinal axis.

12. The heart valve prosthesis of claim 11, wherein the annular frame comprises, at the first axial location along the frame longitudinal axis:
a second frame extension portion at a third circumferential location, the second frame extension portion comprising the first radius, and wherein the second circumferential location and the third circumferential location are spaced circumferentially apart within a range from about 45 degrees to about 75 degrees about the frame longitudinal axis; and
a second frame intermediate portion at a fourth circumferential location, the second frame intermediate portion comprising the second radius, and wherein the third circumferential location and the fourth circumferential location are spaced circumferentially apart within a range from about 45 degrees to about 75 degrees about the frame longitudinal axis.

13. The heart valve prosthesis of claim 12, wherein the annular frame comprises, at the first axial location along the frame longitudinal axis:
a third frame extension portion at a fifth circumferential location, the third frame extension portion comprising the first radius, and wherein the fourth circumferential location and the fifth circumferential location are spaced circumferentially apart within a range from about 45 degrees to about 75 degrees about the frame longitudinal axis; and
a third frame intermediate portion at a sixth circumferential location, the third frame intermediate portion comprising the second radius, and wherein the fifth circumferential location and the sixth circumferential location are spaced circumferentially apart within a range from about 45 degrees to about 75 degrees about the frame longitudinal axis, and wherein the first circumferential location and the sixth circumferential location are spaced circumferentially apart within a range from about 45 degrees to about 75 degrees about the frame longitudinal axis.

14. A method for imparting a shape to an annular frame of a heart valve prosthesis, the method comprising:
positioning a body portion of a mandrel within a lumen of the frame, the body portion extending along a mandrel longitudinal axis between a first end and a second end with a central region extending between the first end and the second end, an outer radial surface of the central region spaced a first radial distance from the mandrel longitudinal axis at a first location and spaced a second radial distance from the mandrel longitudinal axis at a second location that is spaced circumferentially apart from the first location, the first radial distance different than the second radial distance;
contacting the frame with the body portion to impart a shape to the frame such that the frame comprises a first radius at a first circumferential location and a second radius at a second circumferential location, the first circumferential location and the second circumferential location located at the same axial location along a frame longitudinal axis of the frame, the first radius different than the second radius.

15. The method of claim 14, further comprising positioning a cap of the mandrel radially exterior from the frame such that the cap circumferentially surrounds the frame and the body portion with the frame radially between the cap and the body portion.

16. The method of claim 15, further comprising contacting an outer radial surface of the frame with one or more projections of the cap, the one or more projections projecting from an end of the cap toward the first end of the body portion.

17. The method of claim 16, wherein the one or more projections constrain outer radial movement of portions of the frame in contact with the one or more projections, the one or more projections comprising:
a first projection;
a second projection spaced circumferentially apart from the first projection within a range from about 100 degrees to about 140 degrees; and
a third projection spaced circumferentially apart from the second projection within a range from about 100 degrees to about 140 degrees.

18. The method of claim 14, wherein the first radius is greater than the second radius.

19. The method of claim 18, wherein the contacting the frame with the body portion to impart the shape comprises imparting a plurality of frame extension portions comprising the first radius and a plurality of frame intermediate portions comprising the second radius, the plurality of frame extension portions and the plurality of frame intermediate portions located at the same axial location along the frame longitudinal axis.

20. The method of claim 19, wherein a frame extension portion of the plurality of frame extension portions is spaced circumferentially apart from an intermediate portion of the plurality of frame intermediate portions within a range from about 45 degrees to about 75 degrees about the frame longitudinal axis.
